(19) United States
(12) Patent Application Publication
(10) Pub. No.: US 2023/0087224 A1
(43) Pub. Date: Mar. 23, 2023

(54) Adjustable Dose Needleless Injector

(71) Applicant: IDEE INTERNATIONAL R&D INC., Ville-Saint Laurent (CA)

(72) Inventors: Karim MENASSA, Ville Saint-Laurent (CA); Maurice MENASSA, Pierre-fonds (CA); Adam GADOUA, Laval (CA)

(21) Appl. No.: 17/801,737

(22) PCT Filed: Feb. 26, 2021
(86) PCT No.: PCT/CA2021/050242

§ 371 Date: Aug. 23, 2022

Related U.S. Application Data
(60) Provisional application No. 62/982,343, filed on Feb. 27, 2020.

(51) Int. Cl. A61M 5/30; A61M 5/31; A61M 5/315; A61M 5/20

(52) U.S. Cl. CPC A61M 5/30; A61M 5/3129; A61M 5/3155; A61M 5/2053; A61M 2205/583

(57) ABSTRACT

A needle-free injector, and method of using said injector, for injecting variable, discrete doses of liquids intradermally or transdermally into a subject in need thereof, are provided. The injector comprises a barrel having a front section, an intermediate section and a rear section; and a cap having a rear end connectable to the front section of the barrel and rotatable about the front section of the barrel, the cap having a discharge end for receiving a cartridge containing the liquid to be injected, wherein rotation of the cap about a longitudinal axis of the front section of the barrel adjusts the dose of liquid to be discharged from the cartridge by the injector in iscrete dose units. A piston cap is also provided for adjusting the force of the impact of an injector piston on a plunger on the cartridge.

### FIELD

This invention relates to a needleless injector for injecting humans or animals.

### BACKGROUND

There are a great number of patents and other publications describing spring or gas pressure powered needleless (or needle-free) injectors. Many of the apparatus are used to administer a fixed dose or operate at a fixed pressure.

### SUMMARY

The present invention provides a relatively simple needleless (needle-free) injector, which permits easy adjustment of the dose of a liquid, such as a medicament, to be administered, and the pressure of an injection. Thus, the apparatus can be readily adjusted to inject variable, discrete doses of liquid medicament intradermally or transdermally into fat or muscle without the need of an external reservoir such as a syringe or bottle.

In accordance with one aspect, the present application provides a needle-free injector for injecting variable, discrete doses of liquids intradermally or transdermally into a subject in need thereof, the needle-free injector comprising: a barrel having a front section, an intermediate section and a rear section; and a cap having a rear end connectable to the front section of the barrel and rotatable about the front section of the barrel, the cap having a discharge end for receiving a cartridge containing the liquid to be injected; wherein rotation of the cap about a longitudinal axis of the front section of the barrel adjusts the dose of liquid to be discharged from the cartridge by the injector.

In one embodiment, the front section of the barrel comprises a generally tubular body having an externally threaded front end for mating with internal threads of the cap, and the discharge end of the cap comprises threads for mating with threads on the cartridge. The cartridge may also be mounted in the discharge end of the cap using a bayonet coupling.

In one embodiment, the cartridge comprises a generally tubular cartridge body having a discharge end comprising an orifice for discharging liquid therethrough from the cartridge, and a rear end comprising a plunger which is slidably engageable within the barrel of the injector. The cartridge body extends through the cap and a front end of the front section of the barrel.

In one embodiment, the front section of the barrel comprises an opening, preferably having a clear plastic window therein, for allowing a rear end of the plunger to be visible therethrough, such that movement of the plunger frontward or rearward is visible through the opening when the cap comprising the cartridge is rotated about the longitudinal axis of the front section of the barrel. The front section further comprises indicia, preferably located on the clear plastic window, for indicating the amount of the dose to be discharged from the injector when an injection is made.

When the cap is screwed onto, or unscrewed from, the front section of the barrel, a position of the rear end of the plunger moves within the front section and provides an indication of the dose of the liquid to be discharged from the injector. The cap is releasably locked into one or more positions, typically by a locking ring mounted in the rear end of the cap. In one embodiment, the locking ring comprises an annular body with a generally U-shaped cross section the open side of which is outwardly-facing, and an O-ring seated in the annular body.

In one embodiment, balls (preferably plastic balls) are seated in small holes on an inner wall of the annular body, wherein during rotation of the cap, the balls slide around the body of the front section following a helical path of travel, such that, at selected locations, the balls encounter longitudinally extending grooves in the front section, and the O-ring presses the balls into the grooves, releasably locking the cap in position. Rotation of the cap adjusts the dose of liquid to be discharged from the cartridge by the injector is incremental by one or more dose increments, such that increasing the rotation increases the amount of the dose of liquid to be discharged. Preferably, the dose increment can be 0.025 mL or more, but can be modified to other dose increments as needed. The cap is rotated to set the cartridge plunger into a position for injection of the liquid in one or more discrete dose increments. In one embodiment, after the liquid is discharged from the injector, the cap is rotated to reset the cartridge plunger into a position for further injection of the liquid in one or more amounts of discrete dose increments corresponding to the total amount of dose to be administered.

In one embodiment, the injector further comprises a piston cap connected to a threaded rear end of a shaft at a rear barrel section of the injector for adjusting the position of a piston, within a piston assembly, which impacts against the cartridge plunger to discharge liquid from the cartridge, wherein the rotating the piston cap changes the length of the piston stroke, thereby providing an increase or decrease of the force of impact of the piston against the cartridge plunger. The piston assembly comprises the piston, a casing, and a magnet, such that when the piston cap and the shaft are rotated, spacing between a front end of the magnet and casing and the rear end of a connector in communication with the rear end of the plunger, is changed which results in a change in the force imparted to the plunger by the piston. The rear barrel section comprises indicia for providing an indication of the position of the piston and consequently the force to be applied to the piston. Frontward and rearward displacement of the piston is constant at a given setting of the force applied.

In another aspect, the present application provides a method of injecting a liquid from the needle-free injector as described herein, into a subject in need thereof, the method comprising: attaching the cap to the front section of the barrel; inserting a cartridge into the discharge end of the cap, the cartridge comprising a cartridge body, a plunger, and a chamber having a quantity of liquid to be injected, such that the cartridge is attached to the discharge end of the cap; rotating the cap such that the cartridge moves rearwardly in the front section of the barrel, thereby selecting a dose of liquid to be administered; and actuating a trigger on the injector to inject the liquid into the subject. In one embodiment, in the step of rotating the cap, the dose to be administered is selected by the position of the rear end of the plunger which moves beneath an opening, preferably having a clear plastic window therein, and indicia on the window which provide an indication of the dose of the liquid to be discharged from the injector. The steps of rotating the cap and actuating the trigger are repeated for additional doses to be administered, and rotating the cap selects discrete dose units, such that one or more dose units are selectable for each injection. In one embodiment, the method further comprises the step, prior to actuating the trigger, of rotating a piston cap, wherein rotating the piston cap changes the length of the piston stroke, thereby providing an increase or decrease of the force of impact of the piston against the cartridge plunger.

Advantageously, the needle-free injector and method as described herein provide for the administration of one or more adjustable discrete doses of liquid medication from a single cartridge, without the need of multiple injectors or cartridges. This provides for a more convenient and efficient administration of multiple doses of the same medicament, reducing time and costs. It also provides for adjusting the force of impact for injecting the medicament, at a higher or lower force depending on the surface and the subject being injected.

### BRIEF DESCRIPTION OF THE DRAWINGS

Preferred embodiments of the invention are described hereinafter with reference to the accompanying drawings, wherein:

FIG. 1 is a side view of a needleless injector in accordance with the invention;

FIG. 2 is a top view of the injector of FIG. 1;

FIG. 3 is a longitudinal sectional view of the injector taken generally along line 3-3 of FIG. 2;

FIG. 4 is an isometric view of the front section of a barrel used in the injector of FIGS. 1 to 3;

FIG. 5 is a top view of the barrel section of FIG. 4;

FIG. 6 is a side view of the barrel section of FIGS. 4 and 5;

FIG. 7 is a side view of the front end of a plunger used in the injector of FIGS. 1 to 3;

FIG. 8 is a top view of a syringe barrel used in the injector of FIGS. 1 to 3;

FIG. 9 is a front view of the syringe barrel of FIG. 8;

FIG. 10 is a longitudinal sectional view of the syringe barrel taken generally along line 10-10 of FIG. 8;

FIG. 11 is a cross section taken generally along 11-11 of FIG. 8;

FIG. 12 is an isometric view of a bayonet connector used in the injector of FIGS. 1 to 3;

FIG. 13 is a side view of the bayonet connector of FIG. 12;

FIG. 14 is a front view of the bayonet connector of FIGS. 12 and 13;

FIG. 15 is a cross section taken generally along line 15-15 of FIG. 14;

FIG. 16 is a side view of a spacer used with the injector of FIGS. 1 to 3;

FIG. 17 is a rear view of the spacer of FIG. 16;

FIG. 18 is a cross section of the spacer taken generally along line 18-18 of FIG. 17;

FIG. 19 is an exploded, isometric view of piston and stroke adjustment assemblies used in the injector of FIGS. 1 to 3;

FIG. 20 is a longitudinal sectional view of the top, rear end of the injector of FIGS. 1 to 3 on a larger scale;

FIG. 21 is an isometric view of a second embodiment of the needleless injector of the present invention;

FIG. 22 is an exploded, isometric view of the injector of FIG. 21;

FIG. 23 is a longitudinal sectional view of the injector of FIGS. 21 and 22;

FIG. 24 is an isometric view of a front section of a barrel used in the injector of FIGS. 21 and 22;

FIG. 25 is an exploded, isometric view of the front barrel section of FIG. 24;

FIG. 26 is a longitudinal sectional view of the front barrel section of FIG. 24;

FIG. 27 is an isometric view of a disposable syringe used in the injector of FIGS. 21 and 22;

FIG. 28 is a longitudinal sectional view of the syringe of FIG. 27; and

FIG. 29 is an exploded, isometric view of an impactor assembly used in the injector of FIGS. 21 and 22; and

FIG. 30 is a longitudinal sectional view of the top, rear end of the injector of FIGS. 21 and 22 on a larger scale.

FIG. 31 shows a threaded adapter.

FIG. 32 shows a wand-type injector for use with the threaded adapter.

FIG. 33 shows a prospective view of the injector of FIG. 32.

### DETAILED DESCRIPTION

With reference to FIGS. 1 to 3, the needleless injector in one embodiment is in the shape of a pistol and includes an elongated cylindrical barrel indicated generally at 1 with a tubular valve 2 connected to the bottom thereof and a grip 3 extending downwardly from the valve 2. A trigger assembly 4 extends downwardly from the barrel 1 in front of the valve 2 and the handle 3.

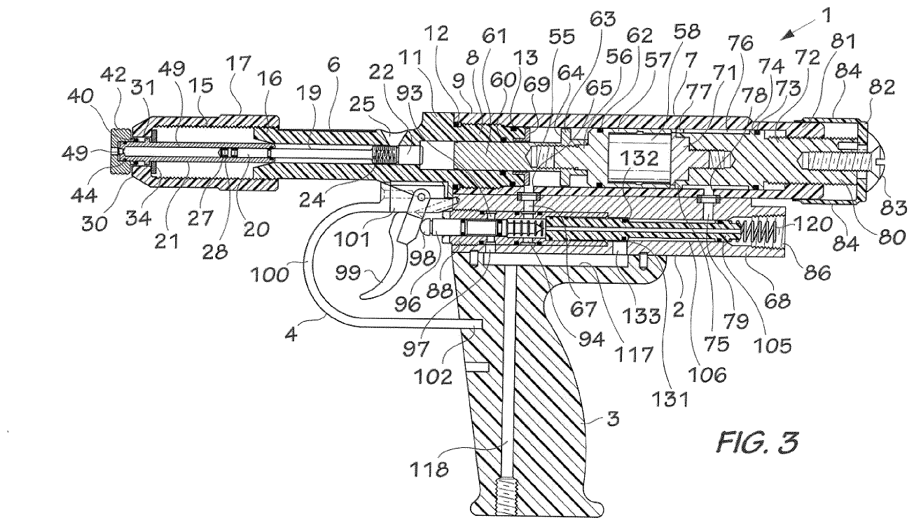

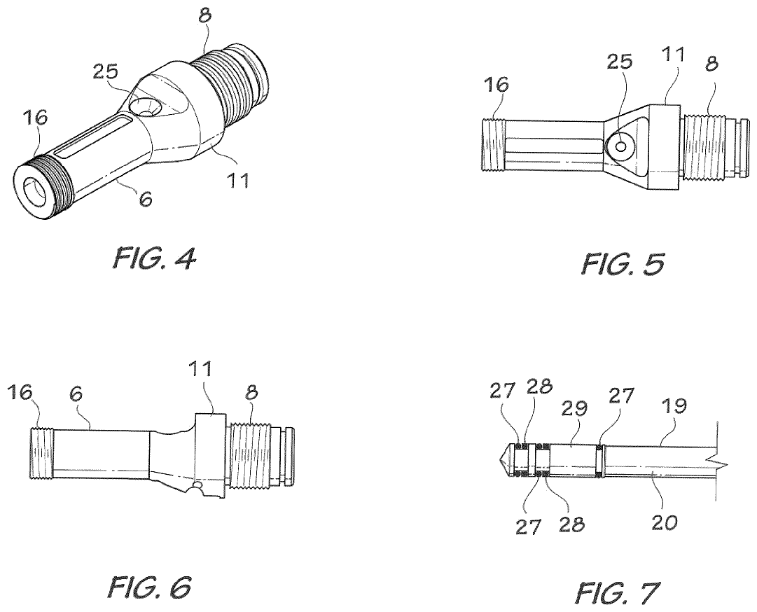

As best shown in FIG. 3, the barrel 1 is defined by front and rear sections 6 and 7, respectively. The externally threaded rear end 8 of the front section 6 mates with the internally threaded front end 9 of the rear section 7. An annular flange 11 on the front section 6 limits movement of the front section into the rear section 7, and O-rings 12 and 13 provide seals between the two sections.

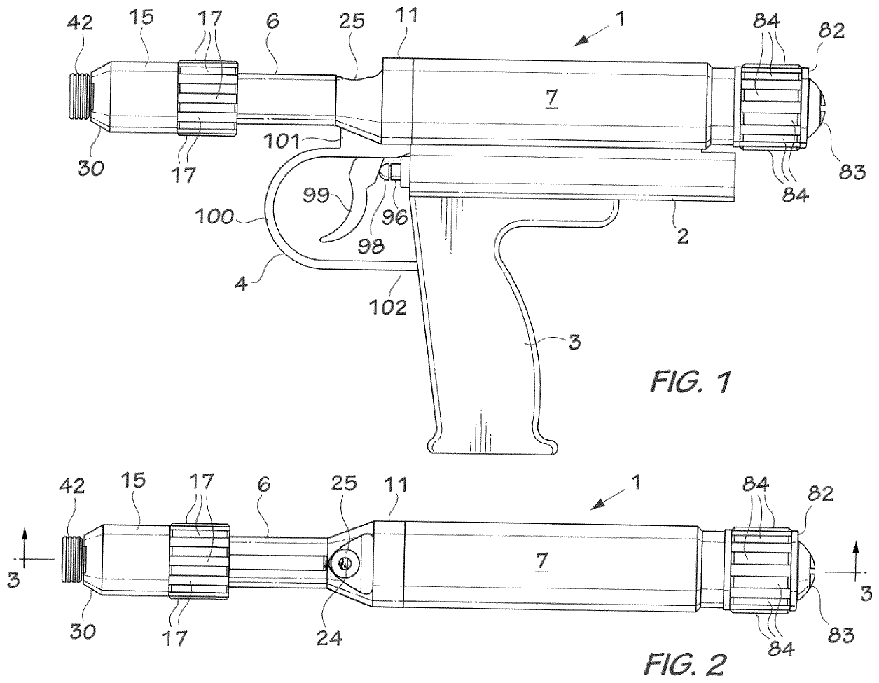

An internally threaded cap 15 is mounted on the externally threaded outer end 16 of the front barrel section 6. Longitudinally extending ridges 17 (FIGS. 1 and 2) on the rear end of the cap 15 facilitate manual rotation of the cap 16 to move it longitudinally relative to the front end of the barrel section 6. The cap 15 is used to adjust the dose of liquid discharged from the injector during an injection. In one embodiment, the doses that can be discharged by the injector may range from 0.025 mL, in increments of 0.025 mL, for example. Other doses and increments may be contemplated, depending on the design of the components of the injector, the force applied against the syringe or cartridge, or the capacity of the syringe or cartridge containing the liquid to be discharged.

A plunger 19 is slidable in the barrel section 6 and in a syringe (or cartridge) barrel 21. The body 20 of the plunger has a large diameter rear end 22 with indicia 24 (FIG. 3) thereon for indicating the dose of liquid to be discharged from the injector. The indicia 24 can be viewed through an opening 25 (FIGS. 2 and 3) in the top of the barrel section 6. When the cap 15 is screwed onto the barrel section 6 or unscrewed therefrom, the position of the rear end 22 of the plunger body 20 moves beneath the opening 25, and the indicia 24 provide an indication of the dose of the liquid to be discharged from the injector. As best shown in FIG. 7, seals including O-rings 27 and TEFLON® rings 28 are provided on the front end 29 of the plunger 19 for sealing the latter in the syringe barrel 21.

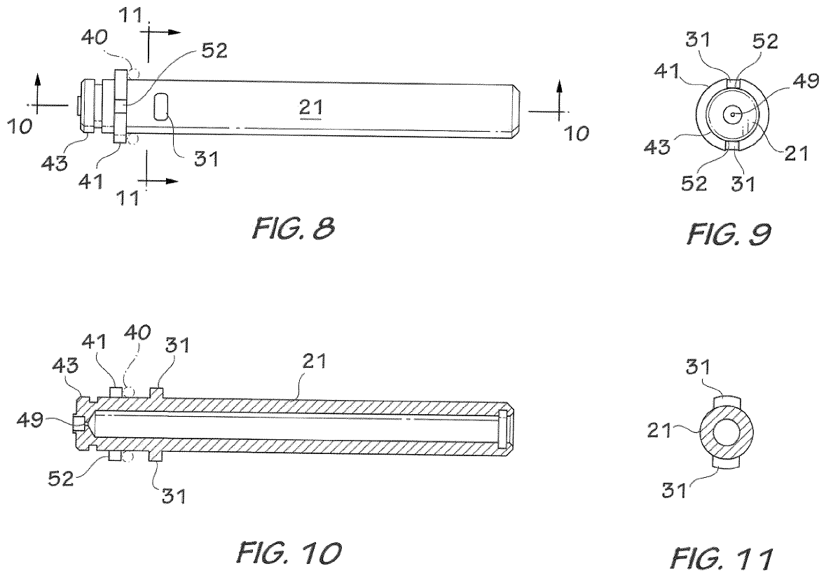

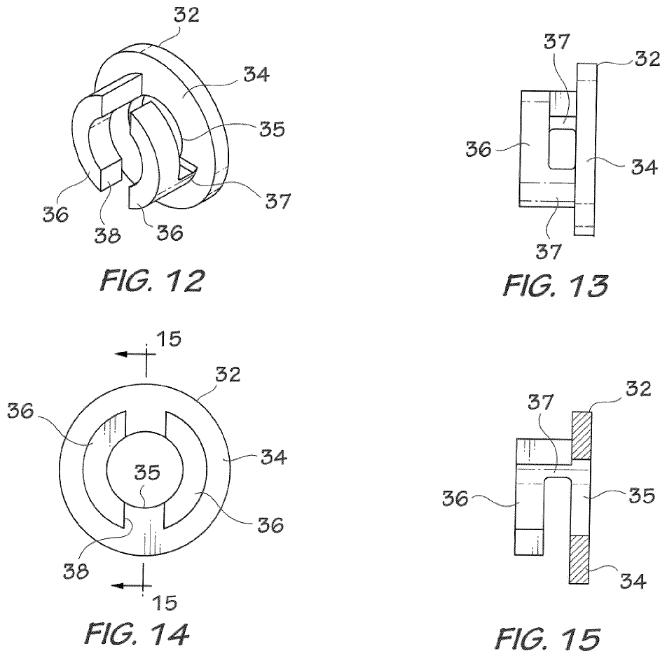

The syringe barrel 21 is mounted in the front end 30 of the cap 15 using a bayonet coupling defined by ears 31 (FIGS. 8, 10 and 11) on the top and bottom of the syringe barrel 21 and a connector 32 mounted in the narrow diameter front end of the cap 15. Referring to FIGS. 12 to 15, the connector 32 includes an annular backplate 34, the central hole 35 of which receives the syringe barrel 21, and a pair of crescent-shaped arms 36 connected by stops 37 at opposite ends to the backplate 34. The arms 36 open towards each other and together define a major part of a ring around and spaced apart from the hole 35. When mounting the syringe barrel 21 in the cap 15, the connector 32 is positioned inside the front end 30 of the cap 15 and the syringe barrel 21 is slid through the opening 35 in the connector backplate until the ears 31 pass through a gap 38 between the arms 36 and abut the backplate 34. Then the syringe barrel 21 is rotated around its longitudinal axis until the ears 31 engage the stops 37. An O-ring 40 is provided between a front flange 41 (FIGS. 8 and 10) on the syringe barrel 21 and the front ends of the connector arms 36. The O-ring 40 acts as a compression spring to prevent movement of the syringe barrel 21 once it is in position.

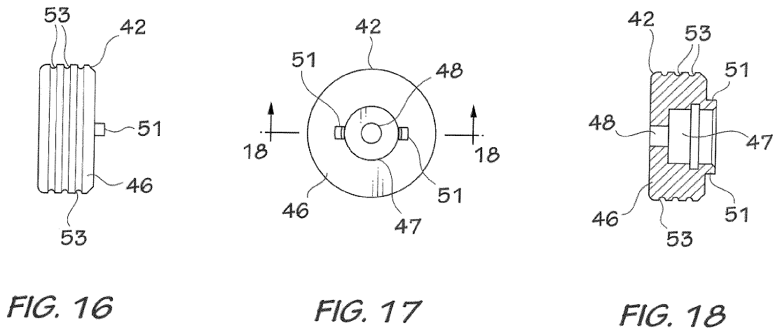

A spacer 42 for intradermal injections can be mounted on the outer, front end 43 of the syringe barrel 21. The spacer 42, is sealed on the outer end 43, of the syringe barrel 21 by an O-ring 44 (FIG. 3). As best shown in FIGS. 16 to 18, the spacer 42 includes a cylindrical body 46 with a passage 47 therethrough for discharging liquid from the barrel 21. The outlet end 48 of the passage 47 is the same diameter as the orifice 49 (FIGS. 1, 9 and 10) at the discharge end 43 of the syringe barrel 21. A pair of lugs 51 on the rear end of the spacer 42 are inserted into notches 52 (FIG. 9) in the front flange 41 on the syringe barrel 21. Annular grooves 53 are provided in the body 46 of the spacer 42 for engagement by an adapter (not shown) on a medicine bottle.

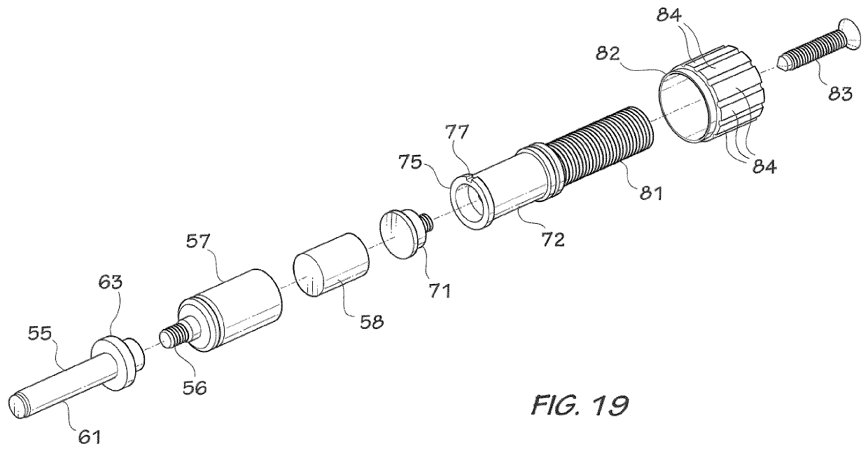

The plunger 19 is driven forward by a piston 54 defined by a retractor 55 (FIGS. 3, 19 and 20) mounted on the threaded neck 56 of a brass casing 57 containing a magnet 58. An O-ring 60 (FIG. 3) seals the cylindrical body 61 of the retractor 55 with respect to the front section 6 of the barrel, and an O-ring 62 seals the magnet casing 57 with respect to the rear section 7 of the barrel 1. An annular flange 63 on the retractor body 61 slides in the barrel 1 during forward movement of the piston 54. When the piston 54 moves forward, gas is expelled through an opening 65 in the bottom of the barrel section 6 into the valve 2 via an opening 67 in the top of the valve body 68. An annular, steel shock absorber 69 is mounted on the rear end of the barrel section 6.

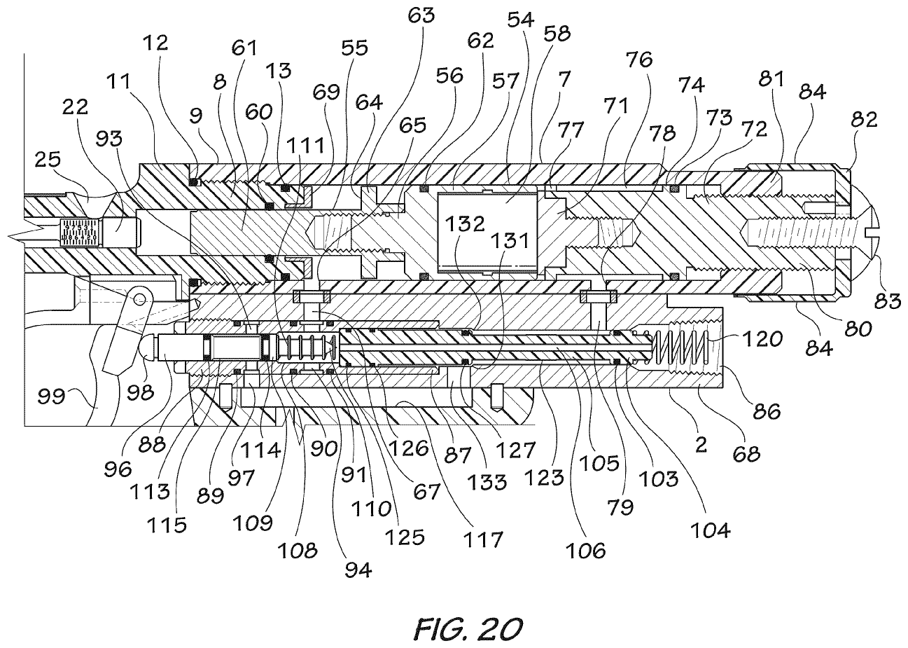

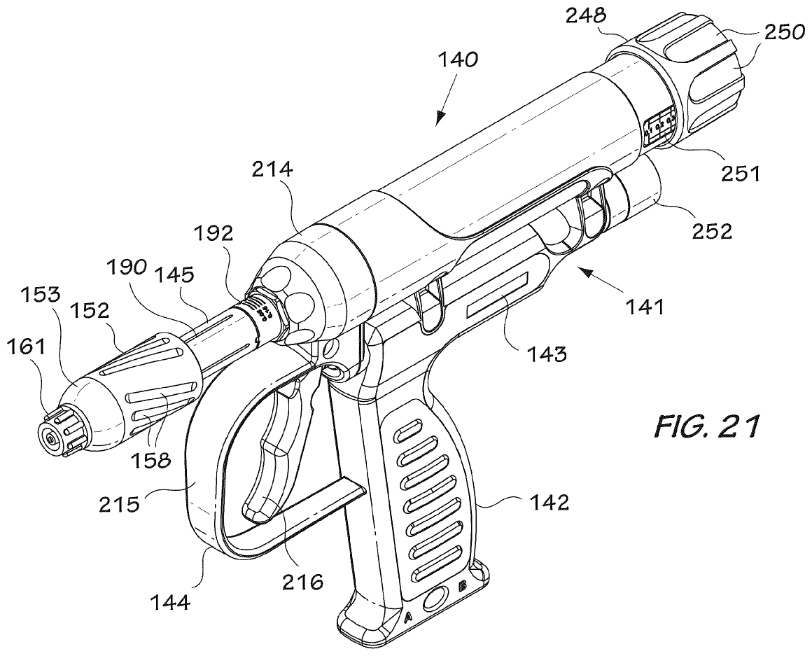

The cylindrical magnet 58 and the casing 57 and consequently the entire piston are held in the rest position (FIG. 3) by a steel retainer 71 mounted on the front end of an adjuster shaft 72. The shaft 72 is sealed in the barrel by an O-ring 73 in an annular flange 74 on the shaft. A flange 75 is also provide on the front end of the shaft 72, the area between the flanges 74 and 75 defining a chamber 76 for receiving gas from the valve 2. Gas entering the chamber 76 passes through a notch 77 in the flange 75 for driving the piston 54 forward. The gas enters the chamber 76 via opening 78 and 79 in the bottom of the barrel 1 and the top of the valve body 68, respectively. As best shown in FIG. 20, the threaded rear end 80 of the shaft 72 extends through the internally threaded open rear end 81 of the rear barrel section 7.

After an injection has occurred, the user resets the cartridge plunger 159 to an injection position by rotating the cap 152 to force the plunger rearwards (away from the discharge end) by an amount corresponding to the dose to be administered. For example, one rotation moves the plunger rearwards to prepare for administration of one dose unit (such as 0.025 mL); for administering multiple discrete increments of the dose unit, the cap 152 is rotated multiple times, as needed. For example, two rotations of the cap 152 on an injector having an incremental dose unit of 0.025 mL, will provide for a total injection of 0.05 mL (i.e., 2×0.025 mL) with one actuation of the injector trigger.

A cap 82 (piston cap) is connected to the threaded rear end 81 (FIGS. 3 and 20) of the shaft 72 by a screw 83 for rotating the shaft. Like the cap 15, the cap 82 includes longitudinally extending ridges 84, which facilitate manual manipulation of the cap. When the cap 82 and the shaft 72 are rotated, the spacing between the front end of the magnet casing 57 and the shock absorber 69 (FIGS. 3 and 20) is changed which results in a change in the length of the piston stroke and consequently the force imparted to the syringe plunger 19 by the piston 54.

Compressed air or another gas under pressure is introduced into the rear end 86 of the valve body 68. As mentioned above, the valve 2 includes the tubular cylindrical body 68 containing the openings 67 and 79 for admitting and discharging a gas under pressure from the chambers 64 and 76 in front of the retractor flange 63 and behind the magnet 58 and the casing 57, respectively. A sleeve 87 with an externally threaded front end 88 is mounted in the internally threaded front end of the valve body 68. The sleeve 87 is sealed in the valve body 68 by O-rings 89, 90 and 91 which border diametrically opposed openings 93 and 94 in the sleeve.

A front valve stem 96 is slidable in the sleeve 87 to permit or prevent access to openings 93 and 97 in the valve body 68. The front valve stem 96 has a hemispherical front end 98 extending out of the sleeve 87 which is engaged by a trigger 99 to push the valve stem 96 rearwardly. As best shown in FIGS. 1 and 3, the trigger 99 forms part of the trigger assembly 4, which also includes a generally U-shaped trigger guard 100. The trigger guard 100 is connected at its top end 101 to the barrel 1 and at its bottom end 102 to the handle 3.

In the rest position of the injector, air is blocked from entering the rear chamber 76 via openings 78 and 79 by an O-ring 103 in a flange 104 at the rear end to of a rear valve stem 105. Air is allowed to pass through a longitudinally extending passage 106 in the rear valve stem 105. The air flows through a chamber 108 bounded by a flange 109 on the rear end of the front valve stem 96 and the front end of the rear valve stem 105, and then passes through openings 67 and 63 into the front chamber 64.

When the front valve stem 96 is pushed rearwardly, the pointed, small diameter rear end 110 (FIG. 20) thereof enters the front end of the passage 106 in a rear valve stem 105 to close the passage. During rearward movement of the front valve stem 96, a helical spring 111 on the rear end 110 is compressed between the flange 109 on the valve stem 96 and the front end of the rear valve stem 105. The front valve stem 96 is sealed in the sleeve 87 by O-rings 113 and 114 in grooves near each end of a reduced diameter central area 115 of the stem 96. As the front stem 96 slides rearwardly, the rear end of the reduced diameter area 115 of the stem 96 moves into alignment with the openings 65 and 67, whereby gas in the front chamber 64 can pass through the openings 65 and 67, along the reduced diameter area 115 and through the bottom of the openings 93 in the sleeve and the opening 97 in the valve body 68 into passages 117 and 118 (FIG. 3) in the handle 3 for venting to the atmosphere.

As the front valve stem 96 moves rearwardly, compressing the spring 111, it pushes the rear valve stem 105 rearwardly against a helical spring 120. Once the flange 104 (FIG. 20) on the rear end of the stem 105 slides into the wide rear, inlet end 86 of the valve body 68, gas under pressure enters a passage 123 around a reduced diameter section of the rear valve stem. The gas flows through the openings 79 and 78, and enters the chamber 76 behind the piston 54. The rear valve stem 109 is sealed in the rear end of the sleeve 87 and in the valve body 68 by O-rings 104, 125 and 126 when in the rest position (FIG. 3) and by O-rings 125, 126 and 127 (FIG. 20) when in an open position, i.e. when gas is flowing into the chamber 76. The gas under pressure entering the chamber 76 drives the piston 54 forward so that the front end of the retractor 55 impacts the rear end 22 of the plunger 19 to expel the contents of the syringe barrel 21. As the piston 54 moves forward, gas is expelled from the chamber 64 in the manner described above. When the trigger 99 is released, the springs 110 and 120 return the front and rear valve stem 96 and 109, respectively to the rest positions shown in FIGS. 3 and 20. When moving to the rest position of the valve stems, gas flowing through the tubular rear stem 109 enters the chamber 64 via the passage 107 in the stem 109, a chamber 130 between the front valve stem flange 112 and the front end of the valve stem 109, and the openings 65 and 94 to drive the piston 54 rearwardly. The magnet 58 comes to rest against and is held in the rest position by the steel retainer 71. As the magnet moves rearwardly, gas is expelled from the chamber 76 via the openings 78 and 79, the passage 123, a gap 131 between a shoulder 132 at the front end of the passage 123, a second opening 133 (FIG. 20) in the bottom of the valve body 68 and passages 117 and 118 in the handle 3.

With reference to FIGS. 21 to 23, the second embodiment of the needleless injector also includes an elongated, cylindrical barrel indicated generally at 140, a tubular valve 141 beneath the barrel 140 and a grip or handle 142 extending downwardly from the valve 141. In this embodiment of the invention, the barrel 140, the body 143 of the valve 141 and the grip 142 are a formed one-piece by injection molding. A trigger assembly 144 extends downwardly from the barrel 140 in front of the valve body 143 and the grip 142.

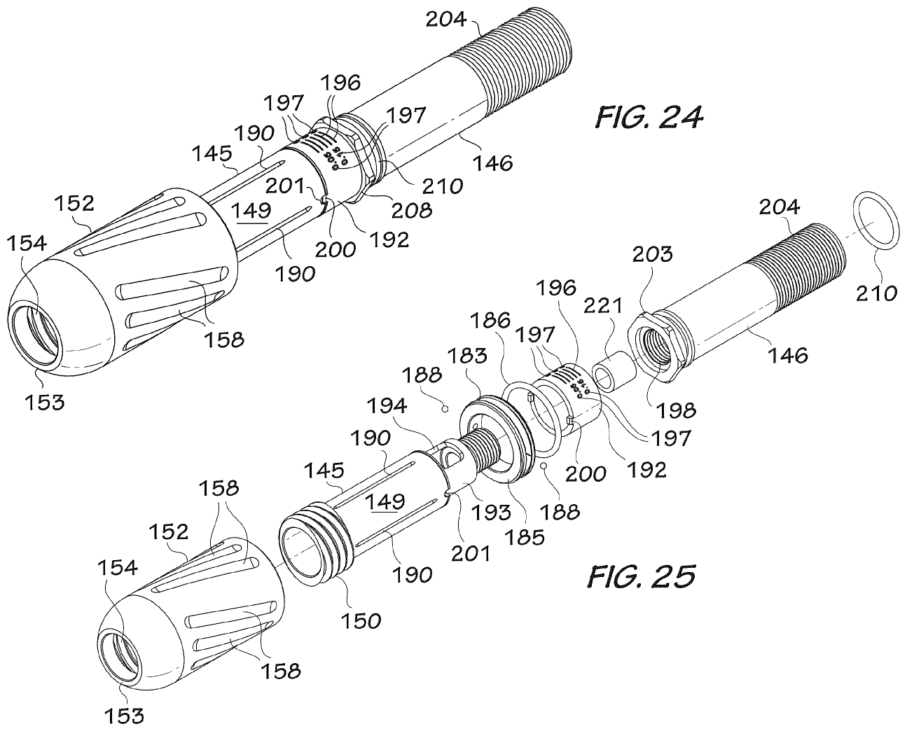

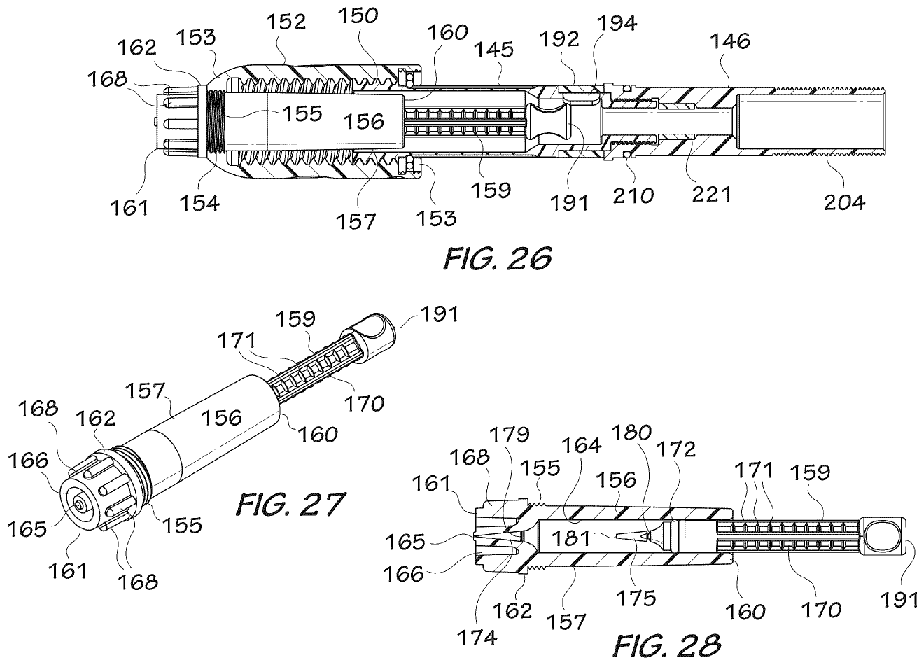

The barrel 140 is defined by a front section 145, an intermediate section 146, a rear section 147 and a tubular connector 148, which connects the intermediate section 146 to the rear section 147. As best shown in FIGS. 24 to 26, the front section 145 of the barrel 140 includes a tubular body 149 with an externally threaded front end 150 for mating with the internally threaded tubular cap 152. The open front, discharge end 153 of the cap 152 includes threads 154 (FIG. 25) for mating with the threads 155 on the tubular body 156 of a disposable syringe or cartridge 157 (FIGS. 27 and 28). Examples of disposable syringes of this type are described in U.S. Pat. No. 9,662,460 to Menassa, incorporated herein by reference). Longitudinally extending grooves 158 in the cap 152 facilitate manual rotation of the cap to move it longitudinally of the front end section 145 of the barrel 140.

The disposable syringe 157 includes the tubular body 156 for slidably receiving a piston or plunger (cartridge plunger) 159. The body 156 has an open inner end 160 and a closed outer end 161. An annular flange 162 near the outer or front end 161 limits movement of the body 156 into the barrel 140. The threads 155 on the body 156 behind the flange 162 engage the internally threaded discharge end 153 of the cap 152 when mounting the syringe in the injector. Fluid, typically medicine, from a chamber 164 in the body 156 is discharged through an orifice 165 in the outer end 161 of the body 156. A recess 166 in the end 161 forms part of a luer lock for connecting a conventional externally threaded needle, catheter or other device (not shown) to the syringe. Longitudinally extending ribs 168 on the cylindrical outer end 161 of the body 156 facilitate gripping of the body when screwing the syringe 157 into the injector barrel 140. The plunger 159 includes an elongated body 170 of cruciform cross section throughout most of its length with reinforcing gussets 171. An O-ring 172 seals the plunger body 170 in the body 156.

As best shown in FIG. 28, the discharge end 174 of the chamber 164 and the corresponding end 175 of the plunger 159 have essentially the same shape. The end 174 of the chamber 164 tapers to the orifice 165 and includes an annular projection or restriction 179. The end 175 of the plunger 159 has a taper identical to that of the end 174 of the chamber 164, and an annular groove 180 (line of weakness) near the tip 181 thereof. A longitudinally extending slot (not shown) can be provided in the end 175 so that the tip can compress when encountering the restriction 179.

In operation, the orifice end of the syringe 157 is connected to a medicine bottle (not shown) and the plunger 159 is retracted to draw medicine into the chamber 164. When the plunger 159 is pushed into the body 156 during an injection, the tip 181 is jammed into the end 174 of the chamber 164, and the projection 179 enters the groove 180. When the plunger 159 is retracted, the narrow tip 181 of the plunger remains in position against the orifice 165 while the remainder of the plunger is retracted. Thus, the orifice 165 is permanently blocked from the inside, preventing re-use of the syringe.

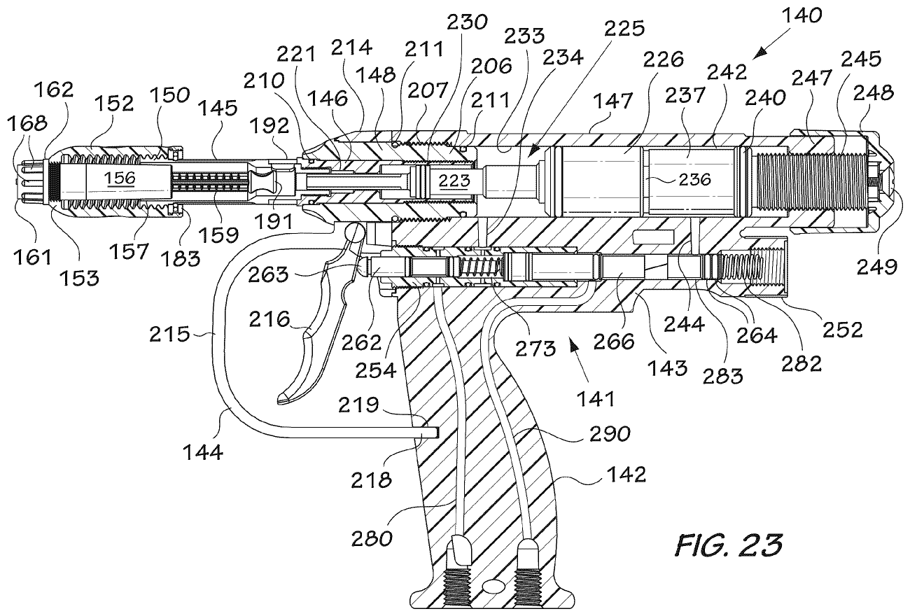

With a loaded syringe 157 mounted in the cap 152, the syringe body 156 extends through the cap 152 and the front end of the front barrel section 145. The cap 152 is releasably locked in one of a plurality of positions by a locking ring 183 (FIGS. 23, 25 and 26) mounted in the rear end of the cap 152. The locking ring 183 has an annular body 185 with a U-shaped cross section with the open side facing outwardly. An O-ring 186 is seated in the body 185. Plastic balls 188 are seated in small holes on the inner wall of the ring body 185. The balls 188 are biased inwardly by the O-ring 186 against the body of the front barrel section 145. During rotation of the cap 152, the balls 188 slide around the body of the front section 145 following a helical path of travel. At selected locations, the balls 188 encounter longitudinally extending grooves 190 (FIG. 25) in the front barrel section 145. The O-ring 186 presses the balls 188 into the grooves 190, releasably locking the cap 152 in position.

When a loaded syringe 157 is mounted in the cap 152, the rear end 191 of the plunger 159 is visible beneath a clear, plastic cylindrical window 192 mounted on a reduced diameter rear end 193 of the front barrel section 145. A hole 194 in the top of the end 193 makes it possible to see the rear end 191 of the syringe plunger 159. Indicia in the form of lines 196 and numbers 197 (FIGS. 24 and 25) on the window 192 provide an indication of the dose that will be discharged from the injector when an injection is made. When the threaded rear end 193 of the front barrel section 145 is mated with the internally threaded front end 198 of the intermediate barrel section 146, the window 192 is sandwiched between the two barrel sections 145 and 146. Ears 200 (FIGS. 24 and 25) extend forwardly from the front end of the window 192 into notches 201 in the rear end of the front barrel section 145 to prevent rotation of the window 192 relative to the front and intermediate barrel sections 145 and 146, respectively.

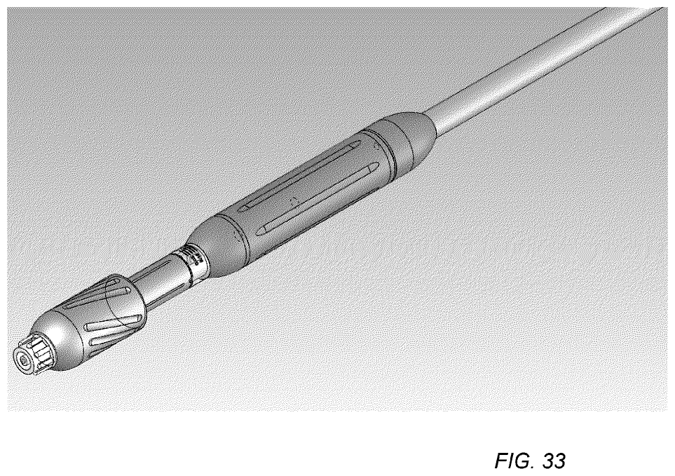

A hexagonal flange 203 on the front end of the intermediate barrel section 146 facilitates rotation of the section during mounting of the section in the connector 148. During mounting, the externally threaded rear end 204 of the intermediate barrel section 146 mates with the internally threaded rear end 206 of the connector 148. In another embodiment, a threaded nut 300 (as shown in FIG. 31) replaces intermediate barrel section 146 and connects with the body of the injector at threaded section 301 of the injector shown (see FIGS. 32 and 33). Threaded nut 300 permits attachment of the front barrel section with other wand-type injectors and attachments such as the control box described, for example, in U.S. Pat. No. 9,067,020, incorporated herein by reference. External threads 207 on the rear end 205 of the connector 148 mate with internal threads on the front end of the rear barrel section 147. O-rings 210 and 211 (FIG. 28) provide seals between these three elements of the injector.

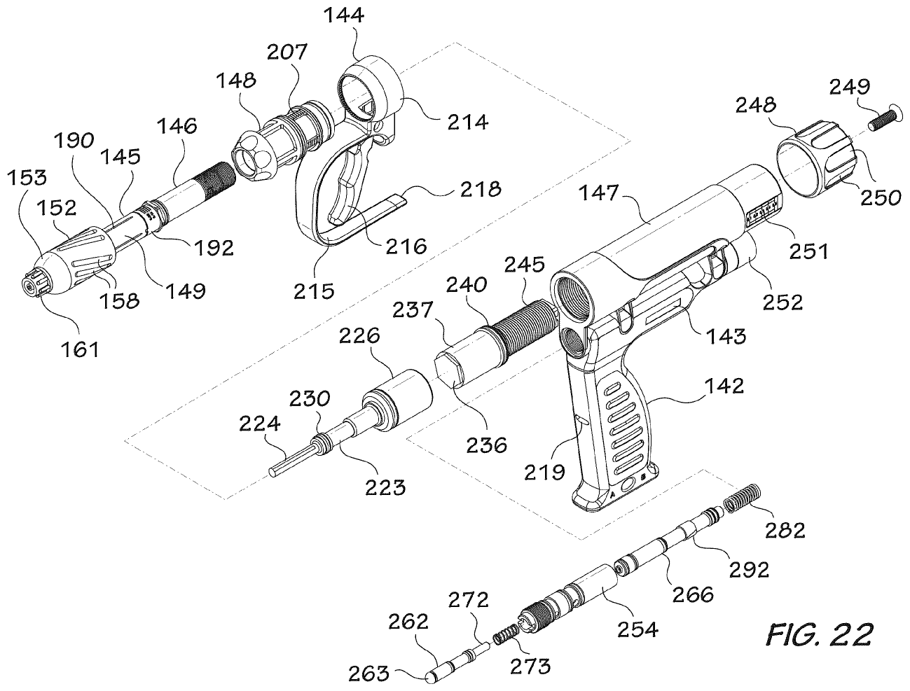

The trigger assembly 144 is mounted on the connector 148 near the front end thereof. The trigger assembly 144 includes a sleeve 214 which slides onto the connector 148, a generally C-shaped trigger guard 215 extending downwardly from the sleeve 214 and a trigger 216 pivotally mounted in the top end of the trigger guard 215. The bottom rear end 218 of the trigger guard 215 extends into a notch 219 (FIGS. 22 and 23) in the front end of the grip 142.

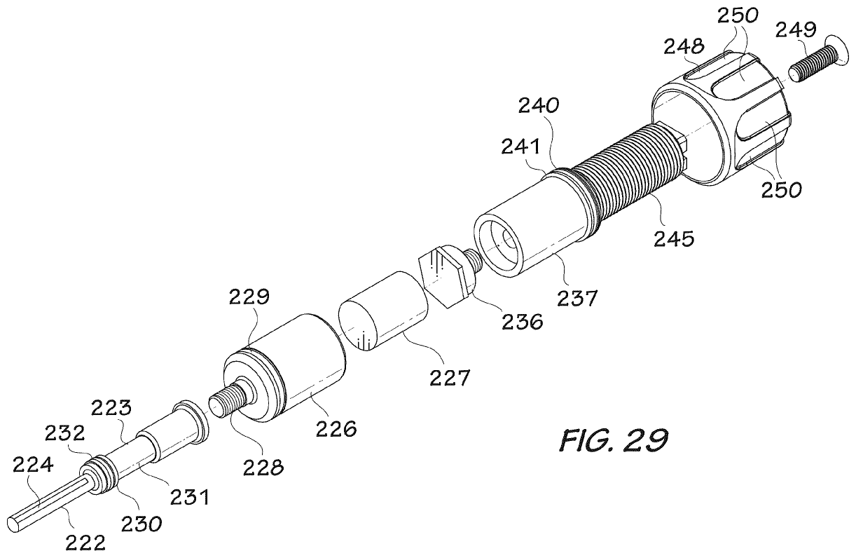

A sleeve bearing 221 is mounted in the intermediate barrel section 146. The reduced diameter front end 222 of a piston 223 slides in the bearing 221 between a retracted position (FIG. 23) and an extended position (not shown) in which it impacts the rear end 191 of the syringe plunger 159. The piston 223 contains three longitudinally extending grooves 224 (FIG. 29) spaced equidistant apart permitting air flow through the bearing 221. The piston 223 defines the front end of a piston assembly 225 (FIGS. 23, 29 and 30).

The syringe plunger 159 is driven forward by the piston assembly 225 defined by the piston 223, a brass casing 226 and a magnet 227 (FIGS. 23, 29 and 30). The piston 223 is mounted on the threaded neck 228 of the brass casing 226. The casing 226 is sealed in the barrel 140 by an O-ring 229. An annular flange 230 defining a piston ring on the piston 223 slides in the barrel 140 during forward movement of the piston. The flange 230 contains an O-ring 232, which provides a seal between the intermediate barrel section 146 and the piston. When the piston 223 moves forward, gas is expelled from the front chamber 233 (FIG. 23) between the rear end of the connector 148 and the front end of the casing 227 through an opening 234 in the bottom of the rear barrel section 147 into the valve 141.

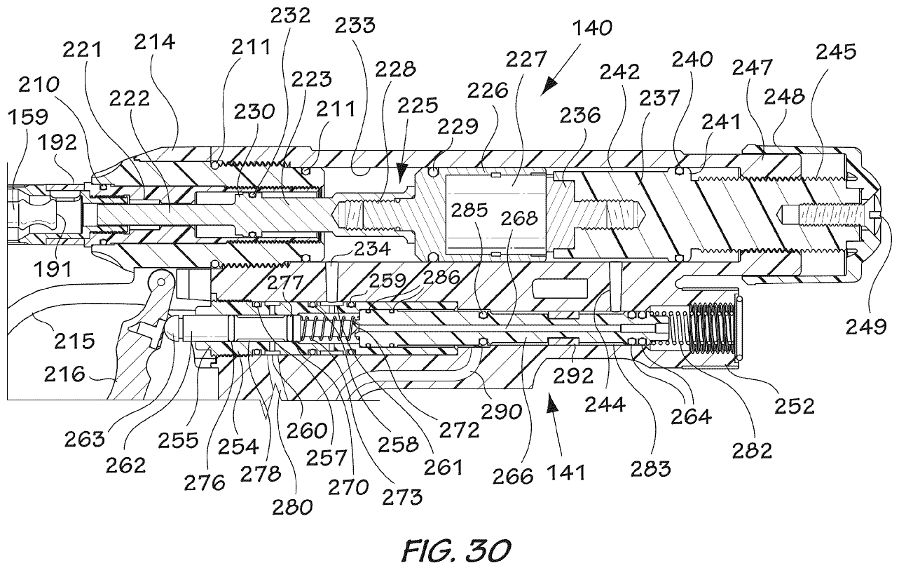

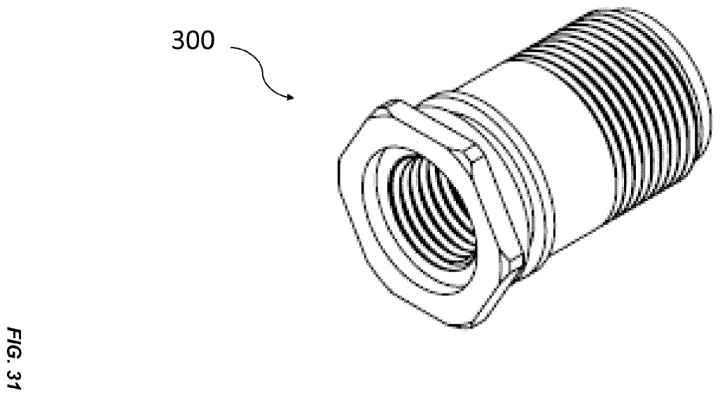

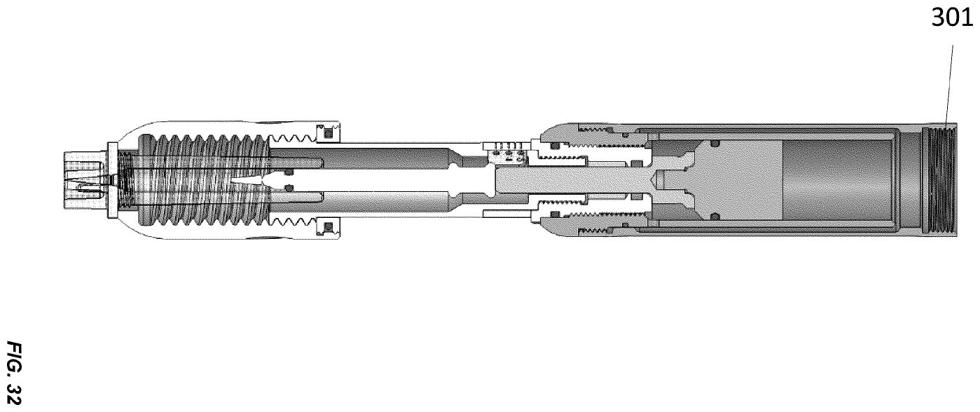

The magnet 227 and the casing 226 are held in the rest position (FIG. 3) by a steel retainer 236 mounted in the front end of an adjuster shaft 237. The shaft 237 is sealed in the barrel 140 by an O-ring 240 in an annular flange 241 on the shaft. The area between the flange 241 and the rear end of the casing 226 defines a chamber 242 for receiving gas from the valve 141. Gas entering the chamber 242 drives the piston assembly forwardly. The gas enters the chamber 242 via opening 244 in the bottom of the barrel 140 and the top of the valve body 143. As best shown in FIG. 30, the threaded rear end 245 of the shaft 237 extends through the internally threaded open rear end 247 of the rear barrel section 147.

A cap 248 (piston cap) is connected to the threaded rear end 247 (FIGS. 23, 29 and 30) of the shaft 237 by a screw 249 for rotating the shaft. Like the cap 152, the cap 248 includes longitudinally extending grooves 250 (FIG. 29), which facilitate manual manipulation of the cap. When the cap 248 and the shaft 237 are rotated, the spacing between the front end of the magnet casing 226 and the rear end of the connector 148 is changed which results in a change in the force imparted to the syringe plunger 159 by the piston 225. Indicia 251 on the side of the barrel 140 provide an indication of the position of the piston 225 and consequently the force to be applied to the piston.

Compressed air or another gas under pressure is introduced into the rear end 252 of the valve body 143. As mentioned above, the valve 141 includes the tubular body 143 containing the openings 234 and 244 for admitting and discharging a gas under pressure from the chambers 233 and 242 in front of the flange 229 and behind the magnet 227 and the casing 226, respectively. A sleeve 254 with an externally threaded front end 255 is mounted in the internally threaded front end of the valve body 143. The sleeve 254 is sealed in the valve body 143 by O-rings 257, 258 and 259 (FIG. 30) which border diametrically opposed openings 260 and 261 in the sleeve.

A front valve stem 262 is slidable in the sleeve 254 to permit or prevent access to the grooves 240 and 241 in the valve body 143. The front valve stem 262 has a hemispherical front end 263 extending out of the sleeve 254 which is engaged by the trigger 216 to push the valve stem 262 rearwardly.

In the rest position of the injector, air is blocked from entering the rear chamber 242 via the opening 244 by O-rings 264 in a flange (FIG. 30) at the rear end of a rear valve stem 266. Air is allowed to pass through a longitudinally extending passage 268 in the rear valve stem 266. Air is free to flow through a chamber 269 bounded by a flange 270 on the rear end of the front valve stem 262 and the front end of the rear valve stem 266.

When the front valve stem 262 is pushed rearwardly by the trigger 216, the pointed, small diameter rear end 272 (FIGS. 22 and 30) thereof enters the front end of the passage 268 in the rear valve stem 266 to close the passage. During rearward movement of the front valve stem 262, a helical spring 273 on the rear end 272 is compressed between the rear end of the valve stem 262 and the front end of the rear valve stem 266. The front valve stem 262 is sealed in the sleeve 254 by O-rings 276 and 277 in grooves near each end of a reduced diameter central area 278 of the stem. As the front stem 262 slides rearwardly, the end of the reduced diameter area 278 of the stem moves into alignment with the opening 234, whereby gas in the front chamber 233 can pass through the opening 234, along the reduced diameter area 278 and through the bottom of the opening 260 in the sleeve 254 and the opening into a passage 280 (FIGS. 23 and 30) in the grip 142 for venting to the atmosphere.

As the front valve stem 262 moves rearwardly, compressing the spring 273, it pushes the rear valve stem 266 rearwardly against a helical spring 282. Once the 0-rings 264 and the flange 265 (FIG. 30) on the rear end of the stem 266 slide into the wide rear, inlet end 252 of the valve body 143, gas under pressure enters a passage 283 around a reduced diameter section of the rear valve stem 266. The gas flows through the opening 244, and enters the chamber 242 behind the piston magnet casing 226. The rear valve stem 266 is sealed in the rear end of the sleeve 254 and in the valve body by O-rings 264, 285 and 286 when in the rest position (FIG. 23) and by O-rings 285, 286 and 288 (FIG. 30) when in an injection position, i.e. when gas is flowing into the chamber 242. The gas under pressure entering the chamber 242 drives the piston 223 forwardly so that the front end impacts the rear end 191 of the syringe plunger 159 to expel the contents of the syringe barrel 157. As the piston 225 moves forward, gas is expelled from the front chamber 233 in the manner described above. When the trigger 216 is released, the springs 273 and 282 return the front and rear valve stem 262 and 266, respectively to the rest positions shown in FIGS. 23 and 30. When the valve stems 262 and 266 are moving to the rest position, gas flowing through the tubular rear stem 266 enters the chamber 233 via the passage 268 in the stem, a chamber between the front valve stem flange 262 and the front end of the valve stem, and the opening 234 to drive the piston 225 to rearwardly. The magnet 227 comes to rest against the steel retainer 236. As the piston 225 moves rearwardly, gas is expelled from the chamber 242 via the opening 244, the passage 283, and passage 290 in the handle 142. A split TEFLON® sleeve 292 on the rear valve stem 266 slows down the exhaustion of gas from the chamber 242 which slows down rearward movement of the piston 225 to the rest position.

The above figures and disclosure are intended to be illustrative and not exhaustive. The description will suggest many variations and alternatives to one of ordinary skill in the art. All such variations and alternatives are intended to be encompassed within the scope of the attached claims. Those familiar with the art may recognize other equivalents to the specific embodiments described herein within, without departing from the spirit and scope thereof.

1. A needle-free injector for injecting variable, discrete doses of liquids, preferably a liquid medicament, intradermally or transdermally into a subject in need thereof, the needle-free injector comprising:
a barrel having a front section, an intermediate section and a rear section; and
a cap having a rear end connectable to the front section of the barrel and rotatable about the front section of the barrel, the cap having a discharge end for receiving a cartridge containing the liquid to be injected;
wherein rotation of the cap about a longitudinal axis of the front section of the barrel adjusts the dose of liquid to be discharged from the cartridge by the injector.

2. The needle-free injector of claim 1, wherein the front section of the barrel comprises a generally tubular body having an externally threaded front end for mating with internal threads of the cap.

3. The needle-free injector of claim 1, wherein the discharge end of the cap comprises threads for mating with threads on the cartridge.

4. (canceled)

5. The needle-free injector of claim 1, wherein the cartridge comprises a generally tubular cartridge body having a discharge end comprising an orifice for discharging liquid therethrough from the cartridge, and a rear end comprising a cartridge plunger which is slidably engageable within the barrel of the injector.

6. (canceled)

7. The needle-free injector of claim 5, wherein the front section of the barrel comprises an opening, preferably having a clear plastic window with indicia thereon for indicating the amount of the dose to be discharged from the injector when an injection is made, for allowing a rear end of the cartridge plunger to be visible therethrough, such that movement of the plunger frontward or rearward is visible through the opening when the cap comprising the cartridge is rotated about the front section of the barrel.

8. (canceled)

9. The needle-free injector of claim 5, wherein when the cap is screwed onto, or unscrewed from, the front section of the barrel, a position of the rear end of the cartridge plunger moves within the front section and provides an indication of the dose of the liquid to be discharged from the injector.

10. The needle-free injector of claim 1, wherein the cap is releasably locked into one or more positions.

11. The needle-free injector of claim 10, wherein the cap is releasably locked by a locking ring mounted in the rear end of the cap comprising an annular body with a generally U-shaped cross section the open side of which is outwardly-facing, and an O-ring seated in the annular body.

12. (canceled)

13. The needle-free injector of claim 11, wherein balls, preferably plastic balls, are seated in small holes on an inner wall of the annular body, wherein during rotation of the cap, the balls slide around the body of the front section following a helical path of travel, such that, at selected locations correlating to an increment of dose of liquid to be discharged, the balls encounter longitudinally extending grooves in the front section, and the O-ring presses the balls into the grooves, releasably locking the cap in position.

14. The needle-free injector of claim 1, further comprising a piston cap connected to a threaded rear end of a shaft at a rear barrel section of the injector for adjusting the position of a piston, within a piston assembly, which impacts against the cartridge plunger to discharge liquid from the cartridge, wherein the rotating the piston cap changes the length of the piston stroke, thereby providing an increase or decrease of the force of impact of the piston against the cartridge plunger, the rear barrel section comprising indicia for providing an indication of the position of the piston and consequently the force to be applied to the piston.

15. The needle-free injector of claim 14, wherein the piston assembly comprises the piston, a casing, and a magnet, such that when the piston cap and the shaft are rotated, spacing between a front end of the magnet and casing and the rear end of a connector in communication with the rear end of the plunger, is changed which results in a change in the force imparted to the plunger by the piston.

16. (canceled)

17. The needle-free injector of claim 15, wherein the frontward and rearward displacement of the piston is constant at a given setting of the force applied.

18. The needle-free injector of claim 1, wherein rotation of the cap adjusts the dose of liquid to be discharged from the cartridge by the injector is incremental by one or more dose increments, such that increasing the rotation increases the amount of the dose of liquid to be discharged.

19. The needle-free injector of claim 18, wherein the dose increment is 0.025 mL.

20. (canceled)

21. The needle-free injector of claim 1, wherein the cap is rotated to set the cartridge plunger into a position for injection of the liquid in one or more discrete dose increments.

22. The needle-free injector of claim 21, wherein after the liquid is discharged from the injector, the cap is rotated to reset the cartridge plunger into a position for further injection of the liquid in one or more amounts of discrete dose increments corresponding to the total amount of dose to be administered.

23. A method of injecting a liquid from the needle-free injector of claim 1, into a subject in need thereof, the method comprising:
attaching the cap to the front section of the barrel;
inserting a cartridge into the discharge end of the cap, the cartridge comprising a cartridge body, a plunger, and a chamber having a quantity of liquid to be injected, such that the cartridge is attached to the discharge end of the cap;
rotating the cap such that the cartridge moves rearwardly in the front section of the barrel, thereby selecting a dose of liquid to be administered wherein rotating the cap selects discrete dose units, such that one or more dose units are selectable for each injection; and
actuating a trigger on the injector to inject the liquid into the subject.

24. The method of claim 23, wherein in the step of rotating the cap, the dose to be administered is selected by the position of the rear end of the plunger which moves beneath an opening, preferably having a clear plastic window therein, and indicia on the window which provide an indication of the dose of the liquid to be discharged from the injector.

25. The method of claim 23, wherein the steps of rotating the cap and actuating the trigger are repeated for additional doses to be administered.

26. (canceled)

27. The method of claim 23, further comprising the step, prior to actuating the trigger, of rotating a piston cap connected to a threaded rear end of a shaft at a rear barrel section of the injector for adjusting the position of a piston, within a piston assembly, which impacts against the cartridge plunger to discharge liquid from the cartridge, wherein rotating the piston cap changes the length of the piston stroke, thereby providing an increase or decrease of the force of impact of the piston against the cartridge plunger.
